(19) United States
(12) Patent Application Publication
(10) Pub. No.: US 2013/0211147 A1
(43) Pub. Date: Aug. 15, 2013

(54) LOW PRESSURE DIMETHYL ETHER SYNTHESIS CATALYST

(71) Applicants: MICHAEL CHEIKY, Thousand Oaks, CA (US); Rajashekharam Malyala, Camarillo, CA (US)

(72) Inventors: MICHAEL CHEIKY, Thousand Oaks, CA (US); Rajashekharam Malyala, Camarillo, CA (US)

(21) Appl. No.: 13/567,991

(22) Filed: Aug. 6, 2012

Related U.S. Application Data
(60) Provisional application No. 61/530,813, filed on Sep. 2, 2011.

(51) Int. Cl. B01J 21/16; C07C 41/09

(52) U.S. Cl. CPC B01J 21/16; C07C 41/09
USPC 568/698; 502/63; 502/68

(57) ABSTRACT

A catalyst and process for synthesis of dimethyl ether from synthesis gas are disclosed. The catalyst and process allow dimethyl ether synthesis at low pressures (below 20 bars) at a conversion rate close to the expected equilibrium rate. The catalyst is a combination of a methanol synthesis catalyst and a methanol dehydration catalyst, wherein the dehydration catalyst is a mixture of dehydration agents which allow optimum production of dimethyl ether.

### REFERENCE TO RELATED APPLICATIONS

This application claims priority to U.S. Provisional Patent Application No. 61/530,813, filed on Sep. 2, 2011, the content of which is incorporated herein by reference in its entirety.

### TECHNICAL FIELD

The present invention relates generally to catalysis, and more particularly to a dimethyl ether synthesis catalyst that operates efficiently at low pressures.

### DESCRIPTION OF THE RELATED ART

Dimethyl ether is a versatile compound capable of being used as a combustion fuel, a cooking fuel, an additive to liquefied propane gas, and an intermediate for the production of other chemical compounds. The basic steps in the dimethyl ether synthesis from synthesis gas are as are as follows:

CO+2H₂→CH₃OH 1)

2CH₃OH→CH₃OCH₃+H₂O 2)

Equilibrium syngas conversion is increased as the methanol formed undergoes dehydration to generate dimethyl ether (DME). The water gas shift reaction (WGS) is also involved as side reaction leading to the formation of carbon dioxide and hydrogen according to the following equation:

CO+H₂O→CO₂H₂ 3)

When all 3 reactions happen in a single reactor the process is known as direct conversion of syngas to DME (STD). In this case the net reaction is:

3CO+3H₂→CH₃OCH₃+CO₂

The rate determining step in the dimethyl ether synthesis process is believed to be the methanol synthesis reaction. Intensive efforts have been made to find suitable catalysts which operate under mild conditions. The original catalysts for methanol synthesis were comprised of ZnO and of ZnO/Cr₂O₃. These catalysts allowed synthesis pressures of 300 to 400 bar and synthesis temperatures of 350° C. starting from synthesis gas. Subsequent work by ICI Corp. led to the development of copper based catalysts, of the form Cu/ZnO/Al₂O₃ and Cu/ZnO/Cr₂O₃, termed low pressure catalysts, which allowed commercial operation in synthesis pressures of 30-90 bars and synthesis temperatures of 220° C. to 300° C. Such a methanol synthesis catalyst coupled with alumina or a zeolite such as ZSM-5 is typically used as a DME catalyst. One such commercial catalyst, for example, is disclosed in U.S. Pat. No. 7,033,972, assigned to JFE Holdings. The catalyst disclosed comprises a methanol synthesis catalyst formed around small sized (200 microns or less) alumina particles. Reaction pressure using this catalyst is typically 50 bars. The capital costs to achieve on largest scale even these “low” pressures can be considerable. It is desirable to find catalysts which enable high efficiency conversion of synthesis gas at even lower pressures (preferably below 20 bar) thus avoiding high capital expenditures and operational costs involved in compressing the synthesis gas.

A small scale study by Tohoku University (Omata et al, Applied Catalysis A: General 262 (2004) 207-214) searched for a low pressure methanol synthesis heterogeneous catalyst tolerant to high CO? concentrations using high throughput combinatorial design reactor. A catalyst containing Cu—Zn—Al—Cr—B—Zr—Ga was found to yield of 270 g MeOH/kg cat/hr at 10 bar and 225° C. for syngas containing 30% CO₂. Takeishi (Biofuels (2010), 1(1), pp. 217-226) reports a conversion efficiency of 5%-15% for a direct DME synthesis from syngas using a Cu—Zn—Al catalyst prepared using a sol-gel method at 16 bar and 220° C. This conversion rate is well below the equilibrium conversion rate expected at the stated pressure and temperature. Several homogeneous methanol synthesis catalysts which operate at low pressures are known. U.S. Pat. No. 4,992,480 discusses a methanol synthesis catalyst operating at 100-150° C. and 7 to 11 bars which utilizes a homogeneous catalyst comprised of a transition metal carbonyl complex such as nickel tetracarbonyl and a methoxide salt, both of which are dissolved in a methanol solvent system. U.S. Pat. No. 5,032,618 discusses a homogeneous methanol synthesis catalyst operable at pressures above 10 bars which uses a copper salt in solution mixed with an alkali metal alkoxide in solution, in a solvent such as methanol and tetrahydrofuran. Prior art does not show heterogeneous catalysts which demonstrate high efficiency (greater than 60% conversion) at pressures lower than 20 bar.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

FIG. 1 is a chart including graphs of calculated equilibrium carbon monoxide conversion to dimethyl ether versus reactor pressure for different temperatures.

FIG. 2 is a schematic illustrating the equipment used in the synthesis of dimethyl ether from synthesis gas.

FIG. 3 is a chart illustrating the results of carbon monoxide conversion using the catalyst of the present invention (CP cat) as a function of reactor temperature. Also shown is a graph of calculated equilibrium carbon monoxide conversion at 11 bar versus temperature.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

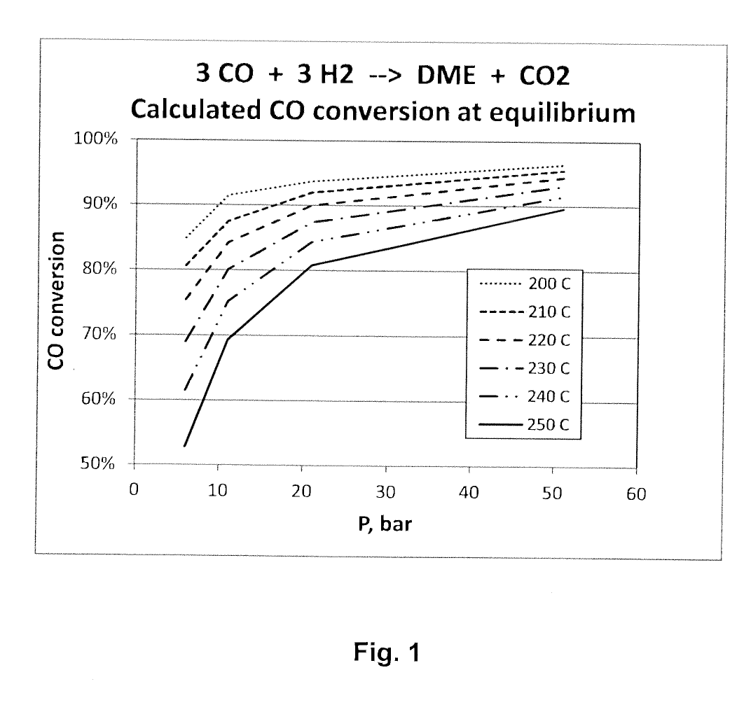

The present invention is directed toward a heterogeneous catalyst which allows efficient syngas conversion to dimethyl ether at pressures lower than those used in present commercial systems. This catalyst comprises a mixture of a methanol synthesis catalyst and a methanol dehydration catalyst, the novelty including a particular selection of methanol dehydration agents with optimum acidity for maximum DME production at low pressures. The difficulty of operating at low pressures is evident from an examination of FIG. 1, which shows calculated equilibrium curves for the conversion of synthesis gas to dimethyl ether as a function of pressure for different temperatures. The conversion rates are shown for temperatures from 200° C. to 250° C. The conversion rates start to decline significantly at pressures below 20 bar. Present commercial catalysts are optimized to work above 30 bar. A catalyst that may be suitable at 50 bar may underperform at pressures below 20 bar. The catalyst of the present invention is optimized to operate at the lower pressures. The full novelty of the invention will become apparent from the following description of the invention.

The methanol synthesis catalysts are well known and comprise co-precipitated oxides of Cu and Zn. These oxides may be co-precipitated with various oxides known to those skilled in the art, including oxides of aluminum, chromium, manganese, zirconium and boron. Typical ratios of Cu to Zn may vary from 5:1 to 1:5. In the case of an aluminum oxide, Al to Cu ratio may vary from 0.05 to 2 and Al to Zn ratio may vary from 0.1 to 1. Co-precipitation may also—performed onto a sol or onto a suspension of well dispersed solid particles. Generally co-precipitation is effected by addition of a basic salt such as sodium carbonate, sodium bicarbonate, ammonium carbonate, or ammonium hydroxide.

After precipitation, the precipitate is filtered, washed and rinsed to remove salt impurities. The clean precipitate is then dried to removal all water and calcined at temperatures from 250° C. to 400° C. The reduced catalyst is believed to comprise Cu crystallites well dispersed on oxygen vacancies in a ZnO matrix. Too high a calcination temperature can cause sintering of the precursor CuO crystallites and reduce catalyst efficiency. The dehydration catalyst, on the other hand, necessitates high calcination temperatures (>400° C.) for the generation of active acid sites, and the dehydration catalyst should be separately calcined from the methanol synthesis catalyst in order to achieve independent activation of both components. After calcination the methanol synthesis powder is further pulverized to attain a suitably large surface area. In some embodiments, the catalyst surface area, as determined via a BET method using nitrogen, should preferably exceed 50 m²/g, and most preferably exceed 100 m²/g.

The dehydration catalyst serves the important role of dehydrating methanol and further pushing the equilibrium synthesis gas conversion. The prior art uses solid acids such as silica alumina, gamma alumina, activated alumina or ZSM-5 to effect this dehydration. Acidity of the catalyst is important for the dehydration reaction. If the acidity of the dehydration catalyst component is low, the resulting catalyst will exhibit low activity as it cannot convert the methanol formed to DME, thereby affecting the equilibrium synthesis gas conversion. If the acidity of the dehydration compound is high, the resulting catalyst will further dehydrate the DME formed to hydrocarbons, thus affecting the production rate of DME. The dehydration component in essence controls the DME selectivity.

Embodiments of the present invention utilize a dehydration catalyst component that is tuned to allow efficient conversion of synthesis gas at pressures below 20 bars. In one embodiment of the invention, the dehydration component is chosen to effect a CO conversion rate exceeding 60% at reaction pressures below 20 bar for temperatures between 220° C. and 300° C. In another embodiment of the invention, the dehydration catalyst component is comprised of a mixture of 2 or more of the following dehydration agents: 20-40% silica alumina, 10 to 30% gamma alumina, 10-50% kaolin, 25%-75% ZSM-5.

In another embodiment of the invention, the dehydration catalyst component is chosen to have an acidity range which optimizes the production of dimethyl ether while minimizing the production of hydrocarbons at pressures below 20 bar. This acidity range corresponds to acidity values lying in between the acidity values of pure gamma alumina and the acidity values of pure ZSM-5. To further test acidities 2.000 g of the following dehydrating agents were titrated with 20% N-butylamine/hexane. While it is recognized that the actual acidity of the catalysts in situ in their dehydrated and/or deammoniated forms may be orders of magnitude higher than at ambient conditions, the butyl amine/hexane room temperature calorimetric titration is expected to correlate with the in situ acidities. The following results were observed:

| Dehydrating Agent | Temp Rise (° C.) | ml titrated |
|---|---|---|
| γ-alumina | 0.538 | 1.8043 |
| Zeolyst ZSM-5 | 1.690 | 2.2085 |
| Silica Alumina Catalyst Support | 1.518 | 1.8049 |
| HZSM-5 + γ-Al2O3 | 1.256 | 2.5148 |
| Silica alumina + γ-Al2O3 | 1.126 | 1.486 |

The temperature rise is an indication of the strength of the acid sites, while the number of milliliters titrated is an indication of the total number of acid sites. Gamma alumina has weakly acidic sites while ZSM-5 has the strong acidic sites compared to the other formulations.

Surprisingly, it has been found that dehydrating agent combinations which produce a butylamine titration temperature rise in the range of 0.8° C. to 1.6° C. are effective dehydrating catalyst components for optimum DME generation for pressures below 20 bar. Suitable acid catalysts for the present invention are heterogeneous (or solid) acid catalysts having one or more solid acidic component. Solid acid catalysts that can be combined include, but are not limited to, (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) inorganic acids or metal salts derived from these acids such as metal sulfides, metal sulfates, metal sulfonates, metal nitrates, metal phosphates, metal phosphonates, metal molybdates, metal tungstates, metal borates, and (7) combinations of groups 1 to 6.

Suitable HPAs include compounds of the general Formula X, M_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Methods for preparing HPAs are well known in the art. Natural clay minerals are well known in the art and include, without limitation, kaolinite, bentonite, attapulgite, montmorillonite and zeolites. When present, the metal components of groups 4 to 6 may be selected from elements from Groups I, IIa, IIIa, VIIa, VIIIa, Ib and IIb of the Periodic Table of the Elements, as well as aluminum, chromium, tin, titanium and zirconium. Fluorinated sulfonic acid polymers can also be used as solid acid catalysts for the process of the present invention. The weight ratio of methanol synthesis component to dehydration component can preferably vary from 5:1 to 1:5, and most preferable from 3:1 to 1:3.

### Example 1

The two components of the dimethyl ether synthesis catalyst were made as follows:

(A) 0.80 moles Cu(NO₃)₂ 0.40 moles Zn(NO₃)₂ and 0.12 moles Al(NO₃)₃ were dissolved in 1.3 L H2O and brought to 80° C. to form Solution A. This solution and 2.5 L of a predissolved 10% aqueous NaHCO3 solution were added dropwise onto a container holding 1 L of water at 80° C. A precipitate is formed as a result of this dropwise addition. The precipitate is aged in the solution for 1 hour, during which time the pH is maintained at 7.0 and the temperature is maintained at 80° C. The resulting precipitate is then filtered and washed with distilled water at 80° C. The clean precipitate is dried at 110° C. for 16 hours. The dried precipitate is calcined at 350° C. for 5 hours. This powder has a BET surface area of 75 m²/g.

(B) The dehydration catalyst was synthesized by mixing 400 g silica alumina catalyst support powder (composition 86% SiO₂, 14% alumina), 250 g gamma alumina, 250 g kaolin, 40 g starch, 20 g lignsulfonic acid, 40 g microcrystalline cellulose with enough water to make an extrudable dough. The extruded dough is dried at 110° C. for 2 hours, calcined at 550° C. for 5 hours, and pulverized to yield catalyst powder with a BET surface area of 275 m²/g.

The powdered methanol synthesis catalyst from A and the dehydration catalyst from B were admixed in a 2:1 ratio with 2% graphite and the admixture was pelletized at 10000 lb/in² to yield a catalyst with a BET surface area of 130 m²/g.

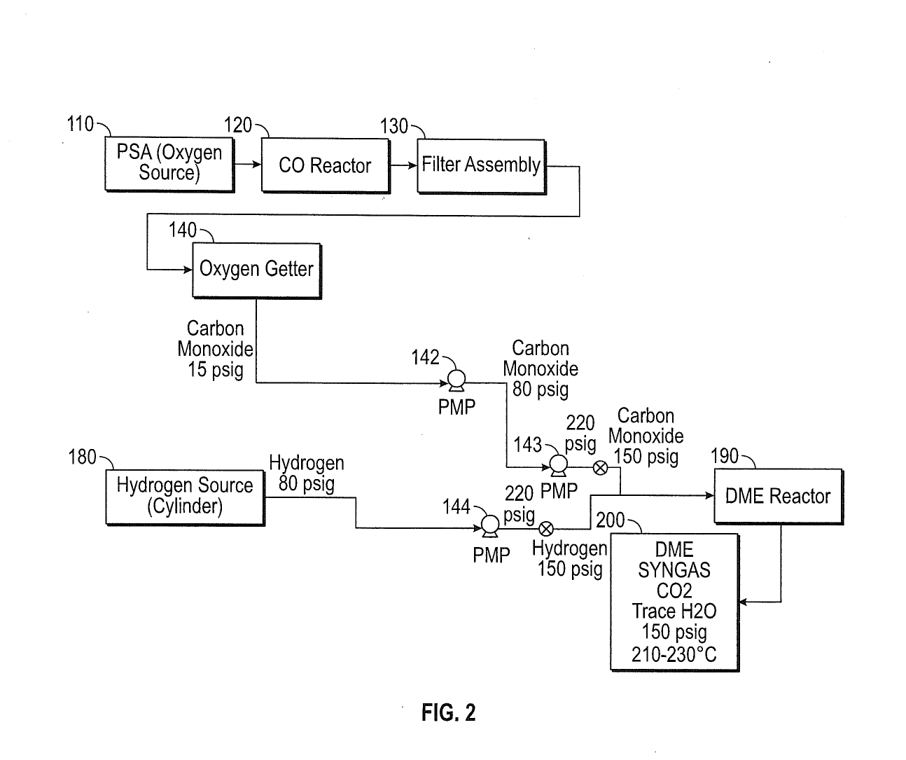

This catalyst was tested in a reactor 190 shown in FIG. 2. The figure shows a schematic of the experimental setup to determine conversion rates from synthesis gas to DME. Carbon Monoxide is generated from reaction of oxygen (after a pressure swing adsorption process 110) with biochar in reactor 120 and passed through filter assembly 130 and oxygen getter 140. The generated carbon monoxide passes through a first pump 142, which compresses it to approximately 80 psig and then to a secondary pump 143, which performs a second compression to 220 psig. Hydrogen is introduced from a cylinder at 40 psig and compressed via pump 144 to 220 psig. Both gases are metered through needle valves into a mixing and preheating chamber, and finally into the catalyst chamber at 150 psig. The reactor temperature is varied between 200° C. and 270° C. at a flow rate space velocity corresponding to 640 hr⁻¹. The input gas composition is H₂/CO/CO₂ 10:9:1.

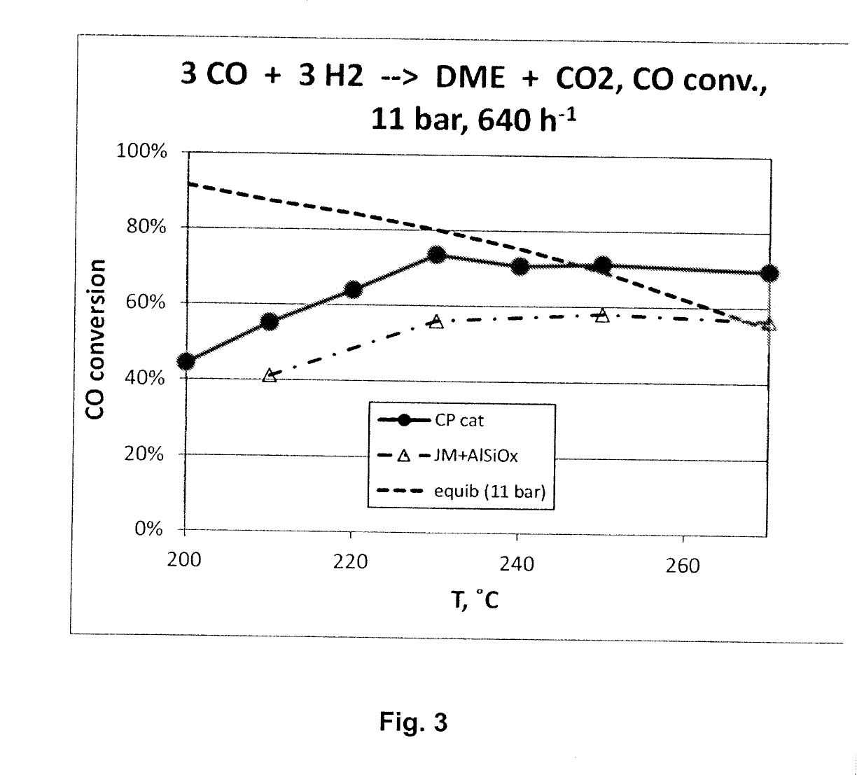

FIG. 3 shows experimental CO conversion results for a catalyst using the method of the present invention (CP cat), a commercial catalyst admixed with silica alumina (JM+AlSiOx) and calculated equilibrium results for various temperatures at 11 bar reaction pressure. It is evident that for temperatures exceeding 230° C. the experimental results of the CP catalyst are a significant improvement over the commercial catalyst, and more closely approximate the equilibrium values, thus indicating an effective catalyst under these conditions.

Modifications may be made by those skilled in the art without affecting the scope of the invention.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. These illustrations and their accompanying description should not be construed as mandating a particular architecture or configuration.

1. A catalyst composition for the synthesis of dimethyl ether from synthesis gas, comprising:
a methanol synthesis component comprising co-precipitated metal components containing Cu, Zn and Al, wherein an atomic ratio of Al to Cu is 0.05 to 2 and an atomic ratio of Al to Zn is 0.1 to 1; and
a dehydration component comprising a mixture of dehydrating agents selected from at least two of the group consisting of: silica alumina, kaolin, gamma alumina, aluminum silicate, montmorillonite, mullite, mesostructured aluminosilicate, and zeolites;
wherein the dehydration component is separately calcined from the methanol synthesis component and the dehydrating agents are selected to yield a CO conversion rate to dimethyl ether exceeding 60% at reaction pressures below 20 bar at temperatures above 220° C. and below 300° C.

2. The catalyst composition according to claim 1, wherein in which a weight ratio of methanol synthesis component to dehydration component varies from 5:1 to 1:5.

3. The catalyst composition according to claim 1, wherein the dehydrating component is calcined at temperatures exceeding 500° C.

4. The catalyst composition according to claim 1, wherein the methanol synthesis component is calcined at temperatures below 400° C.

5. The catalyst composition according to claim 1, wherein a silica alumina concentration varies from 10% to 60% and a kaolin concentration varies from 10% to 50%.

6. The catalyst composition according to claim 1, wherein a silica alumina concentration varies from 10% to 60%, a kaolin concentration varies from 10% to 40%, and a gamma alumina concentration varies from 10% to 50%.

7. The catalyst composition according to claim 1, wherein a silica alumina concentration varies from 10% to 60% and a gamma alumina concentration varies from 10 to 50%.

8. The catalyst composition according to claim 1, wherein a zeolite concentration varies from 25% to 75%, a kaolin concentration varies from 10% to 50% and a gamma alumina concentration varies from 10% to 50%.

9. The catalyst composition according to claim 1, wherein the dehydration component is produced using pore former materials selected from the group consisting of: microcrystalline cellulose, starch, lignocellulosic compounds, acrylates, carboxylases, and sulfonates.

10. The catalyst composition according to claim 1, wherein the dehydration agents cause a temperature rise between 0.8° C. and 1.6° C. when 2.000 g of the agents is calorimetrically titrated against a 20% buty amine/hexane solution.

11. A method of producing dimethyl ether from synthesis gas comprising hydrogen and carbon monoxide, the method comprising:
contacting the synthesis gas with a catalyst;
wherein the catalyst comprises:
a methanol synthesis component comprising co-precipitated metal components containing Cu, Zn and Al, wherein an atomic ratio of Al to Cu is 0.05 to 2 and an atomic ratio of Al to Zn is 0.1 to 1; and
a dehydration component comprising a mixture of dehydrating agents selected from at least two of the group consisting of silica alumina, kaolin, gamma alumina, aluminum silicate, montmorillonite, mullite, mesostructured aluminosilicate, and zeolites;
wherein the dehydration component is separately calcined from the methanol synthesis component and the dehydrating agents are selected to yield a CO conversion rate to dimethyl ether exceeding 60% at reaction pressures below 20 bar at temperatures above 220° C. and below 300° C.

12. The method according claim 11, wherein a weight ratio of methanol synthesis component to dehydration component varies from 5:1 to 1:5.

13. The method according to claim 11, wherein the dehydrating component is calcined at temperatures exceeding 500° C.

14. The method according to claim 11, wherein the methanol synthesis component is calcined at temperatures below 400° C.

15. The method according to claim 11, wherein a silica alumina concentration varies from 10% to 60% and a kaolin concentration varies from 10% to 50%.

16. The method according to claim 11, wherein a silica alumina concentration varies from 10% to 60%, a kaolin concentration varies from 10 to 40%, and a gamma alumina concentration varies from 10 to 50%.

17. The method according to claim 11, wherein a silica alumina concentration varies from 10% to 60% and a gamma alumina concentration varies from 10 to 50%.

18. The method according to claim 11, wherein a zeolite concentration varies from 25% to 75%, a kaolin concentration varies from 10% to 50% and a gamma alumina concentration varies from 10% to 50%.

19. The method according to claim 11, wherein the dehydration component component is produced using pore former materials selected from the group consisting of: microcrystalline cellulose, starch, lignocellulosic compounds, acrylates, carboxylates, sulfonates.

20. The method according to claim 11, wherein the dehydration agents cause a temperature rise between 0.8° C. and 1.6° C. when 2.000 g of the agents is calorimetrically titrated against a 20% butylamine/hexane solution.
